(19) United States
(12) Patent Application Publication
(10) Pub. No.: US 2018/0230545 A1
(43) Pub. Date: Aug. 16, 2018

(54) Method for the prediction of progression of bladder cancer

(71) Applicant: Stichting Katholieke Universiteit, Nijmegen (NL)

(72) Inventors: Antoine G. van der Heijden, Nijmegen (NL); Lourdes Mengual Brichs, Barcelona (ES)

(73) Assignee: Stichting Katholieke Universiteit, Nijmegen (NL)

(21) Appl. No.: 15/750,508

(22) PCT Filed: Aug. 4, 2016
(86) PCT No.: PCT/EP2016/068673

§ 371 Date: Feb. 6, 2018

(30) Foreign Application Priority Data
Aug. 6, 2015 (EP) 15180037.2

(51) Int. Cl. C12Q 1/6886

(52) U.S. Cl. CPC C12Q 1/6886; C12Q 2600/158; C12Q 2600/118

(57) ABSTRACT

The invention relates to the field of medicine, specifically the field of diagnosis and treatment of cancer.

### FIELD OF THE INVENTION

The invention relates to the field of medicine, specifically the field of diagnosis and treatment of cancer.

### BACKGROUND OF THE INVENTION

Urothelial carcinoma is the most common bladder cancer (BC) with an incidence of approximately 90%. At diagnosis two-third of urothelial carcinomas are non-muscle invasive and one-third are muscle invasive. Non-muscle invasive bladder cancer (NMIBC) is characterized by a high risk of recurrence (30-85% depending on stage and grade) after transurethral resection of the bladder tumor (TURBT). Moreover, up to 17% of all NMIBC will eventually progress to muscle invasive bladder cancer (MIBC). Progression is mainly seen within 48 months after initial diagnosis and depends on stage, grade and presence of concomitant Carcinoma In Situ (CIS) [1]. Progressive patients deserve careful attention, particularly because the treatment and pathogenesis of MIBC and NMIBC differ and conventional histopathological evaluation is adequate for diagnosis but is inadequate to accurately predict the behavior of high risk NMIBC. Thus, there is a clear need for predictive biomarkers that can distinguish progressive from non-progressive NMIBC. Given the heterogeneity of BC it is unlikely that a single marker can identify its progression. This underlines the importance of biomarker combinations.

### SUMMARY OF THE INVENTION

In an aspect, the invention provides for a method for the prediction of progression of bladder cancer in a subject comprising determination of the expression level of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 in a sample from a subject, wherein differential expression of at least one, two, three, four or five marker genes is predictive for progression of the bladder cancer.

In a further aspect, the invention provides for the use of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 for predicting, preferably in vitro or ex vivo progression of bladder cancer.

In a further aspect, the invention provides for a method of treatment comprising:
establishing a prediction of progression of bladder cancer in a subject using a method according to the first aspect of the invention, and,
treating a subject wherein the bladder cancer is predicted to progress for progressive bladder cancer, or,
   treating a subject wherein the bladder cancer is predicted not to progress for non-progressive bladder cancer.

In a further aspect, the invention provides for a composition comprising at least one, two, three, four or five sets of nucleic acid molecules suitable for the determination of the expression level of a bladder cancer marker gene selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7.

In a further aspect, the invention provides for a kit useful for performing a method for the prediction of progression of bladder cancer in a subject comprising determination of the expression level of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 in a sample from a subject, comprising at least one, two, three, four or five sets of nucleic acid molecules suitable for the determination of the expression level of a bladder cancer marker gene selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7.

### DETAILED DESCRIPTION OF THE INVENTION

As set forward here above, in non-muscle invasive bladder cancer (NMIBC) conventional histopathological evaluation is inadequate to accurately predict progression to muscle invasion. It is therefore an object of the invention to provide a method to predict tumor progression, especially to predict progression to muscle invasion in subjects suffering from T1G3 bladder cancer.

Surprisingly, it has now been demonstrated that different expression patterns, already at a time point before any clinical evidence, of a new set of genes are predictive for progression and non-progression of T1G3 bladder cancer later on during the disease.

Accordingly, in a first aspect the invention provides a method for the prediction of progression of bladder cancer in a subject comprising determination of the expression level of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 in a sample from a subject, wherein differential expression of at least one, two, three, four or five marker genes is predictive for progression of the bladder cancer. Said method is herein further referred to as a method according to the invention. In the embodiments of the invention, reference is made to genes, preferably human genes, such as but not limited to GUSB, PPIA, ANXA10, DAB2, HYAL2, MAP4K1 and SPOCD1, as marker genes for prediction of progression of bladder cancer; these genes are referred to by their assigned names. The person skilled in the art is able to identify and use these genes for the embodiments of the invention based on these assigned names and to determine, using means known in the art, the expression level of the these genes. In addition, the person skilled in the art may use the sequence data provided in the sequence listing and Table 1 of the application. Preferably, in the embodiments of the invention, at least two bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 are used; more preferably, at least three bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 are used; even more preferably at least four bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 are used; more preferably, at least five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 are used.

In the embodiments of the invention, the bladder cancer is preferably a T1G3 bladder cancer.

In the embodiments of the invention, the expression level of a bladder cancer marker gene, or any other gene, may be determined using any means known to the person skilled in the art, including but not limited to determining the amount of mRNA of a subject gene in a sample and determining the amount of gene product, such a polypeptide, in a sample. The expression level is preferably normalized in view of the expression level of a housekeeping gene, i.e. a gene whose expression is not influenced by the disease or condition of the subject. Of course, it is preferred that the expression level of the subject gene or genes and of the housekeeping gene or genes are determined by identical techniques under identical conditions from the same sample.

In the embodiments of the invention, the term “sensitivity” is preferably defined as the method's ability to identify positive results; in the context of the invention “sensitivity” is thus an indication for the prediction whether in a subject suffering from bladder cancer the cancer will progress.

In the embodiments of the invention, the term “specificity” is preferably defined as the method's ability to identify negative results; in the context of the invention “sensitivity” is thus an indication for the prediction whether in a subject suffering from bladder cancer the cancer will not progress.

In the embodiments of the invention, the term “Positive Predictive Value” (PPV) is defined as the method's ability to identify the proportion of test results that are true positives; in the context of the invention PPV is thus an indication for the proportion of subjects that is correctly predicted as wherein the bladder cancer will actually progress. In the embodiments of the invention, the term “Negative Predictive Value” (NPV) is defined as the method's ability to identify the proportion of test results that are true negatives; in the context of the invention “NPV” is thus an indication for the proportion of subjects that is correctly predicted as wherein the bladder cancer will actually not progress.

Preferably in a method and other embodiments according to the invention, the differential expression is determined:
by comparing the expression level of the bladder cancer marker gene in a sample from a subject suffering from bladder cancer to the expression level of the marker gene in a corresponding sample from a subject not suffering from bladder cancer, or,
by comparing the expression level of the bladder cancer marker gene in a sample from a subject suffering from bladder cancer to an expression level reference value of the bladder cancer marker gene that is indicative for progression of the bladder cancer, or,
by comparing the expression level of the bladder cancer marker gene in a sample from a subject suffering from bladder cancer to an expression level reference value of the bladder cancer marker gene that is indicative for non-progression of the bladder cancer, or,
by comparing the expression level of the bladder cancer marker gene in a sample from a subject suffering from bladder cancer to the expression level of the bladder cancer marker gene in a corresponding sample from a subject suffering from bladder cancer that later in time demonstrated progression of the bladder cancer (i.e. a progressive subject), or
by comparing the expression level of the bladder cancer marker gene in a sample form a subject suffering from bladder cancer to the expression level of the bladder cancer marker gene in a corresponding sample from a subject suffering from bladder cancer that later in time did not demonstrate progression of the bladder cancer (i.e. a non-progressive subject).

Preferably, in the methods and other embodiments according to the invention, the differential expression is determined by comparing the expression level of the bladder cancer marker gene in a sample from a subject suffering from bladder cancer to an expression level reference value of the bladder cancer marker gene that is indicative for progression of the bladder cancer.

As is depicted here above, a reference value in the methods and other embodiments according to the invention may be a reference value obtained from a single sample or subject of may be the average, mean or median of several values obtained from multiple samples of a single or multiple subjects.

The subject expression level of at least one, two, three, four or five bladder cancer marker genes according to the invention in the methods and other embodiments according to the invention may be a value obtained from a single sample of the subject suffering from bladder cancer of may be the average, mean or median of several values obtained from multiple samples of the subject.

Preferably in the methods and other embodiments according to the invention, the expression level is preferably normalized in view of the expression level of an endogenous control gene such as a housekeeping gene, i.e. a gene whose expression is not influenced by the disease or condition of the subject. Accordingly, in the methods and other embodiments according to the invention, prediction of progression of bladder cancer in a subject comprising determination of the expression level of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 in a sample from a subject, preferably further comprises determination of the expression level of an endogenous control gene such as a housekeeping gene, preferably selected from the group consisting of GUSB and PPIA.

Differential expression is herein defined as a statistically relevant difference (preferably p≤0.05) in expression level of a subject gene in a measured sample compared to the expression level of the subject gene measured in another sample, e.g. the expression level of a subject gene in a sample from a subject suffering from bladder cancer compared to the expression level of the subject gene in a sample from a subject not suffering from bladder cancer. Differential expression may be up-regulation (i.e. a higher expression level) or down-regulation (i.e. a lower expression level) and may be depicted as a percentage change or difference, as a fold change or difference, as a log change difference or the like. Preferably, in the methods and other embodiments according to the invention, differential expression of a bladder cancer marker gene is defined as at least 10%, 20%, 30%, 50%, 60%, 70%, 80%, 90%, 100%, 150%, 200%, 300%, 400%, 500%, 600%, 700% or at least 800% up-regulation or down-regulation, compared to the expression level of the corresponding bladder cancer marker gene in a corresponding sample from a non-progressive subject, the expression level of the corresponding bladder cancer marker gene in a corresponding sample from a subject not suffering from bladder cancer, the expression level reference value of the bladder cancer marker gene that is indicative for non-progression of the bladder cancer, or the expression level reference value of the bladder cancer marker gene that is indicative progression of the bladder cancer.

In the methods and other embodiments of the invention, preferably, the progression of bladder cancer is the progression from non-muscle-invasive bladder cancer to muscle-invasive bladder cancer, more preferably the progression from non-muscle-invasive T1G3 bladder cancer to muscle-invasive T1G3 bladder cancer.

The person skilled in the art knows how to discriminate between non-muscle-invasive bladder cancer (NMIBC) to muscle-invasive bladder cancer (MIBC) e.g. by conventional histopathological evaluation.

In the methods and other embodiments of the invention, preferably, the expression level of at least two bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 is determined wherein one of the at least two bladder cancer marker genes is SPOCD1. More preferably, the expression level of at least SPOCD1 and ANXA10 is determined; the expression level of at least SPOCD1 and DAB2 is determined; the expression level of at least SPOCD1 and HYAL2 is determined; or the expression level of at least SPOCD1 and MAP4K1 is determined. Even more preferably, the expression level of at least SPOCD1, ANXA10 and DAB2 is determined; the expression level of at least SPOCD1, ANXA10 and HYAL2 is determined; the expression level of at least SPOCD1, ANXA10 and MAP4K1 is determined; the expression level of at least SPOCD1, DAB2 and HYAL2 is determined; the expression level of at least SPOCD1, DAB2 and MAP4K1 is determined; or the expression level of at least SPOCD1, HYAL2 and MAP4K1 is determined. Even more preferably, the expression level of DAB2, HYAL2, MAP4K1 and SPOCD1 is determined; the expression level of ANXA10, HYAL2, MAP4K1 and SPOCD1 is determined; the expression level of ANXA10, DAB2, MAP4K1 and SPOCD1 is determined; or the expression level of ANXA10, DAB2, HYAL2 and SPOCD1 is determined. Most preferably, the expression level of at least ANXA10, DAB2, HYAL2, MAP4K1 and SPOCD1 is determined.

In addition to the determination of the expression levels of the genes and sets of genes depicted here above, in the methods and other embodiments of the invention, the expression level of an additional bladder cancer marker gene may be determined, including but not limited to a gene selected from the group consisting of: ABAT, ABCC5, ABLIM1, ACAD9, ADSS, AP3M1, ATG3, BCAS1, BIRC4, BIRC5, BIRC6, BNC2, BNIP3L, C15orf52, C16orf74, C6orf136, CCDC75, CCDC80, CDC20, CDC25B, CLN3, CSRP2, DICER1, DUOX1, ELF3, ERGIC2, FAM174B, FGF11, FGFR3, FLJ36031, GAB2, GBA2, GGPS1, GHR, GPRC5A, GRB7, HES5, HIST1H2AH, HIST2H4B, ICMT, IGDCC4, IGF2, IL6ST, LRRC16, MBD6, MCM7, MDK, MUTYH, MXRA7, MYL3, NCKAP1, NRG4, ODC1, OPA1, PCOLCE1, PDPK1, PDPN, PFKFB4, PIK3R1, PLAG1, PPARD, PROC, PRRG2, PSD4, PSIP1, RB23, RAB25, RBP1, RBPMS2, RFX1, RNF216, SCN5A, SERPINB5, SORBS1, STAT5B, TAGLN, THRSP, TMEM1219, TPM1, TRAF4, TTC39C, ULK1, WBP1, WHSC1, ZAK, ZDHHC7 and ZNF816A; more preferably a gene selected from the group consisting of: BNC2, BNIP3L, C16orf74, DAB2, FGFR1, FLJ36031, HES5, HIST1H2AH, HIST2H4B, HYAL2, MAP4K1, MUTYH, PFKFB4, PPARD, SERPINB5, WBP1 and ZDHH7.

In the methods and other embodiments according to the invention, the sample may any relevant sample and may be a direct sample or may be a derived or a processed sample. Preferably, the sample comprises tumor cells, more preferably the sample is a tumor sample, or a bladder fluid sample; preferably the bladder fluid sample is a bladder wash or urine. Samples may be obtained in invasive and non-invasive ways. A preferred non-invasive sample is a bladder fluid, such as a bladder wash or urine.

In the methods and other embodiments according to the invention determination of the expression level of a bladder cancer marker gene, or any other gene, may be determined using any means known to the person skilled in the art, including but not limited to determining the amount of mRNA of a subject gene in a sample and determining the amount of gene product, such a polypeptide, in a sample. Preferably, the determination of the expression level is performed by a nucleic acid amplification method, preferably by PCR, more preferably by RT-PCR, more preferably multiplex PCR or multiplex RT-PCR. PCR primers in the methods and embodiments according to the invention can readily be designed and produced by the person skilled in the art and/or by a service provider; commercially available primers can be used, such as preferably the primers used in the examples herein and listed in Table 1.

Preferably, the method according to the invention is an in vitro or ex vivo method. When a sample is obtained in a non-invasive way, the method will inherently be an in vitro method. When a sample is obtained in an invasive way, the method may still be an ex vivo method since the provision of the sample may be excluded from the method according to the invention.

In a second aspect, the invention provides for the use of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 for predicting, preferably in vitro or ex vivo progression of bladder cancer. In this aspect of the invention, the features and embodiments of the first aspect of the invention are preferred features and embodiments.

In a third aspect, the present invention provides for a method of treatment comprising:
establishing a prediction of progression of bladder cancer in a subject using a method according to the first aspect of the invention, and,
treating a subject wherein the bladder cancer is predicted to progress for progressive bladder cancer, or,
treating a subject wherein the bladder cancer is predicted not to progress for non-progressive bladder cancer.

Said method of treatment is herein referred to as a method of treatment according to the invention. Preferably, in a method of treatment according to the invention, treatment of a subject wherein the bladder cancer is predicted to progress comprises cystectomy, and/or wherein treatment of a subject wherein the bladder cancer is predicted not to progress comprises intravesical therapy such as immune therapy using BCG and/or interferon, or chemotherapy preferably using mitomycin, epirubicin, thiotepa, valrubicin, doxorubicin, and/or gemcitabine, and/or hypothermia.

This aspect of the invention also provides for the use of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 for the preparation of a medicament for the treatment of bladder cancer comprising:
establishing a prediction of progression of bladder cancer in a subject using a method according to the first aspect of the invention, and,
treating a subject wherein the bladder cancer is predicted to progress for progressive bladder cancer, or,
treating a subject wherein the bladder cancer is predicted not to progress for non-progressive bladder cancer.

This aspect of the invention also provides for at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7 for use in an in vivo diagnostic method for the prediction of progression of bladder cancer in a subject using a method according to the first aspect of the invention.

In this aspect of the invention, the features and embodiments of the first aspect of the invention are preferred features and embodiments.

In the methods and other embodiments of the invention, treatment of bladder cancer any be any kind of treatment known to the person skilled in the art, such as e.g. described in Guidelines on Non-muscle-invasive Bladder Cancer (TA, T1 and CIS), Bajuk et al, 2015.

In a fourth aspect, the invention provides for a composition comprising at least one, two, three, four or five sets of nucleic acid molecules suitable for the determination of the expression level of a bladder cancer marker gene as defined the first aspect of the invention, preferably suitable for determination by a nucleic acid amplification method, preferably by PCR, more preferably by RT-PCR, more preferably multiplex PCR or multiplex RT-PCR.

In this aspect of the invention, the features and embodiments of the first aspect of the invention are preferred features and embodiments.

In a fifth aspect, the invention provides for a kit useful for performing the method according to the first aspect of the invention, comprising at least one, two, three, four or five sets of nucleic acid molecules suitable for the determination of the expression level of a bladder cancer marker gene as defined in any of claims 1-10, preferably suitable for determination by a nucleic acid amplification method, preferably by PCR, more preferably by RT-PCR.

In this aspect of the invention, the features and embodiments of the first aspect of the invention are preferred features and embodiments.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified bases. The skilled person is capable of identifying such erroneously identified bases and knows how to correct for such errors. In the embodiments of the invention, reference is made to genes, preferably human genes, such as but not limited to GUSB, PPIA, ANXA10, DAB2, HYAL2, MAP4K1 and SPOCD1, as marker genes for prediction of progression of bladder cancer; these genes are referred to by their assigned names. The person skilled in the art is able to identify and use these genes for the embodiments of the invention based on these assigned names and to determine, using means known in the art, the expression level of the these genes. In addition, the person skilled in the art may use the sequence data provided in the sequence listing and in Table 1 of the application. The person skilled in the art is aware that mutations, whether silent or non-silent may be present in the genes, mRNA and polypeptide sequences provided in herein such as in Table 1 and in the sequence listing. The person skilled in the art knows how to determine the expression level of a gene that is slightly different than the one listed herein or in the prior art. Accordingly, where the expression level of a given gene is described herein, it is to be construed as also comprising the expression level of a gene sequence, mRNA or gene product that has at least 80%, 85%, 90%, 91%, 92%, 93%, 94%, 95%, 96%, 97%, 98%, or 99% sequence identity with the given gene sequence.

All patent and literature references cited in the specification are hereby incorporated by reference in their entirety.

TABLE 1
Sequences

| SEQ | | |
|---|---|---|
| ID | | |
| NO | Gene/Polypeptide | Sequence |
| 1 | ANXA10 gDNA | See sequence listing |
| 2 | ANXA10 mRNA | See sequence listing |
| 3 | ANXA10 protein | See sequence listing |
| 4 | BNC2 gDNA | See sequence listing |
| 5 | BNC2 mRNA | See sequence listing |
| 6 | BNC2 protein | See sequence listing |
| 7 | BNIP3L gDNA | See sequence listing |
| 8 | BNIP3L mRNA | See sequence listing |
| 9 | BNIP3L protein | See sequence listing |
| 10 | C16orf74 gDNA | See sequence listing |
| 11 | C16orf74 mRNA | See sequence listing |
| 12 | C16orf74 protein | See sequence listing |
| 13 | DAB2 gDNA | See sequence listing |
| 14 | DAB2 mRNA | See sequence listing |
| 15 | DAB2 protein | See sequence listing |
| 16 | FGFR1 gDNA | See sequence listing |
| 17 | FGFR1 mRNA | See sequence listing |
| 18 | FGFR1 protein | See sequence listing |
| 19 | FLJ36031 gDNA | See sequence listing |
| 20 | FLJ36031 mRNA | See sequence listing |
| 21 | FLJ36031 protein | See sequence listing |
| 22 | HIST1H2AH gDNA | See sequence listing |
| 23 | HIST1H2AH mRNA | See sequence listing |
| 24 | HIST1H2AH protein | See sequence listing |
| 25 | HYAL2 gDNA | See sequence listing |
| 26 | HYAL2 mRNA | See sequence listing |
| 27 | HYAL2 protein | See sequence listing |
| 28 | MAP4K1 gDNA | See sequence listing |
| 29 | MAP4K1 mRNA | See sequence listing |
| 30 | MAP4K1 protein | See sequence listing |
| 31 | PFKFB4 gDNA | See sequence listing |
| 32 | PFKFB4 mRNA | See sequence listing |
| 33 | PFKFB4 protein | See sequence listing |
| 34 | PPARD gDNA | See sequence listing |
| 35 | PPARD mRNA | See sequence listing |
| 36 | PPARD protein | See sequence listing |
| 37 | SERPINB5 gDNA | See sequence listing |
| 38 | SERPINB5 mRNA | See sequence listing |
| 39 | SERPINB5 protein | See sequence listing |
| 40 | SPOCD1 gDNA | See sequence listing |
| 41 | SPOCD1 mRNA | See sequence listing |
| 42 | SPOCD1 protein | See sequence listing |
| 43 | ZDHHC7 gDNA | See sequence listing |
| 44 | ZDHHC7 mRNA | See sequence listing |
| 45 | ZDHHC7 protein | See sequence listing |
| 46 | GUSB gDNA | See sequence listing |
| 47 | GUSB mRNA | See sequence listing |
| 48 | GUSB protein | See sequence listing |
| 49 | PPIA gDNA | See sequence listing |
| 50 | PPIA mRNA | See sequence listing |
| 51 | PPIA protein | See sequence listing |

| Gene | TaqMan primer probe set from Life Technologies |
|---|---|
| BNC2 | Hs00417700_m1 |
| DAB2 | Hs01120074_m1 |
| FGFR1 | Hs00915142_m1 |
| FLJ36031 | Hs00703139_s1 |
| HIST1H2AH | Hs00544732_s1 |
| HYAL2 | Hs00186841_m1 |
| SPOCD1 | Hs00375905_m1 |
| ZDHHC7 | Hs00938102_m1 |
| SERPINB5 | Hs00985285_m1 |
| BNIP3L | Hs00188949_m1 |
| PPARD | Hs04187066_g1 |
| PFKFB4 | Hs00190096_m1 |
| C16orf74 | Hs00293326_m1 |
| ANXA10 | Hs00200464_m1 |
| MAP4K1 | Hs00179345_m1 |
| PPIA | Hs99999904_m1 |
| GUSB | Hs99999908_m1 |

### FIGURE LEGENDS

FIG. 1.

Study Outline.

Tissue samples were obtained from a total of 40 progressive and 56 non-progressive high risk NMIBC patients. Samples were split in a screening (21 samples) and validation phase (75 samples). Genes differentially expressed between progressive and non-progressive high risk NMIBC patients were identified in the screening phase by using gene expression microarray. Genetic classifiers for NMIBC diagnosis and prediction of tumour aggressiveness were identified, which resulted in a five gene expression signature. None of the samples from the screening set were employed for the validation process. In addition, an internal cross-validation strategy was performed on the proposed signature.

Abbreviations: prog=progressive; non-prog=non-progressive; AUC=Area Under Curve.

FIG. 2.

Differentially expressed genes between progressive and non-progressive high risk NMIBC.

A. Screen dump of heat map displaying the 50 most differentially expressed genes between progressive and non-progressive samples. B. Screen dump of heat map displaying the 75 genes differentially expressed genes between progressive and non-progressive samples to be further analyzed by RT-qPCR.

Red pixels correspond to an increased abundance of mRNA in the samples, whereas green pixels indicate decreased mRNA levels. Rows represent genes and columns represent experimental samples.

FIG. 3.

ROC analysis based on the predicted probabilities derived from a two, three, four gene model and from the five-gene model (3 a, c, e and g) and LOOCV analysis based on the predicted probabilities derived from a two, three, four gene model from the five-gene model (3 b, d, f and h).

### EXAMPLES

The invention is further described by the following examples which should not be construed as limiting the scope of the invention.

Unless stated otherwise, the practice of the invention will employ standard conventional methods of molecular biology, virology, microbiology or biochemistry. Such techniques are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); Oligonucleotide Synthesis (N. Gait editor); Nucleic Acid Hybridization (Hames and Higgins, eds.).

### Example 1. A Five-Gene Expression Signature to Predict Progression in T1G3 Bladder Cancer

### SUMMARY

### Background

In non-muscle invasive bladder cancer (NMIBC) conventional histopathological evaluation is inadequate to accurately predict progression to muscle invasion.

Objective:

To analyze tumour gene expression profiles of progressive and non-progressive T1G3 bladder cancer (BC) patients to develop a gene expression signature to predict tumour progression.

Design, Setting and Participants:

Retrospective, multicenter study of 96 T1G3 BC patients who underwent a transurethral resection. Patients with concomitant CIS were excluded. Formalin-fixed paraffin embedded tissue samples were collected.

Outcome Measurements and Statistical Analysis:

Global gene expression patterns were analyzed in 21 selected samples from progressive and non-progressive T1G3 BC patients using Illumina microarrays. Expression levels of 94 genes selected based on microarray data and based on literature were studied by quantitative PCR in 75 progressive and non-progressive T1G3 BC patients. Univariate logistic regression was used to identify individual predictors. A variable selection method was used to develop a multiplex biomarker model. Discrimination of the model was measured by ROC curve AUC.

Results and Limitations:

Expression levels of 1294 genes differed more than 1.5-fold between progressive and non-progressive patients. Differential expression of fifteen genes was validated by quantitative PCR in an additional set of samples. A five-gene expression signature (ANXA10, DAB2, HYAL2, SPOCD1 and MAP4K1) discriminated progressive and non-progressive T1G3 BC patients with a sensitivity of 79% and a specificity of 86% (AUC=0.83). Limitations are sample size and retrospective design

### INTRODUCTION

Gene expression analyses have had an important effect on the discovery of new molecular markers for diagnosis and prediction of disease outcome in BC [2-4]. In this work differentially expressed genes between progressive and non-progressive T1G3 BC were studied. Subsequently, the recently introduced approach of Biomark Fluidigm Arrays based on the quantitative PCR, allowed validation of identified differentially expressed genes in a larger series. A five-gene signature was identified to predict progression in T1G3 BC patients.

### Materials and Methods

### Patients and Samples

A retrospective multicenter study in which a total of 96 patients (mean age 69 yr, range 39-90 yr; 24 females, 72 males) with T1G3 urothelial carcinoma of the bladder who underwent TURBT in two different centers (Hospital Clinic, Barcelona, Spain and Radboud University Medical Center, Nijmegen, The Netherlands) between 1993 and 2010 were included. Patients with concomitant CIS were excluded. Two groups of patients were formed; non-progressive patients consisting of recurrent non-progressive patients with at least two years of follow up with one or more non-muscle invasive recurrences (N=56) and progressive patients consisting of individuals with a period of at least more than six months between the initial TURBT showing T1G3 BC and the progression to MIBC (N=40). Cytology and urethrocystoscopy three months after the initial TURBT had to be negative; otherwise these patients were qualified as having residual and not progressive disease. The median time to progression was 11 months (range 6 to 81 months). Demographic and clinicopathological characteristics of enrolled patients are shown in Table 3 [5,6]. Tissue samples were obtained under institutional review board-approved protocol.

### Tissue Specimens and RNA Isolation

Upon obtainment the tissue was fixed in 10% formalin within 24 h and subsequently embedded in paraffin. A slide of each specimen was stained with haematoxylin-eosin to determine the presence of tumour. The tumour area was macro-dissected from slides (total thickness 80-μm) and total RNA was isolated using the RecoverAll Total Nucleic Acid Isolation Kit for FFPE (Ambion, Inc. Austin, Tex., USA) according to the manufacturer's protocol. Total RNA was quantified by spectrophotometric analysis at 260 nm (NanoDrop Technologies, Wilmington, Del., USA).

### Whole Genome Gene Expression Microarray: Global Screening Phase

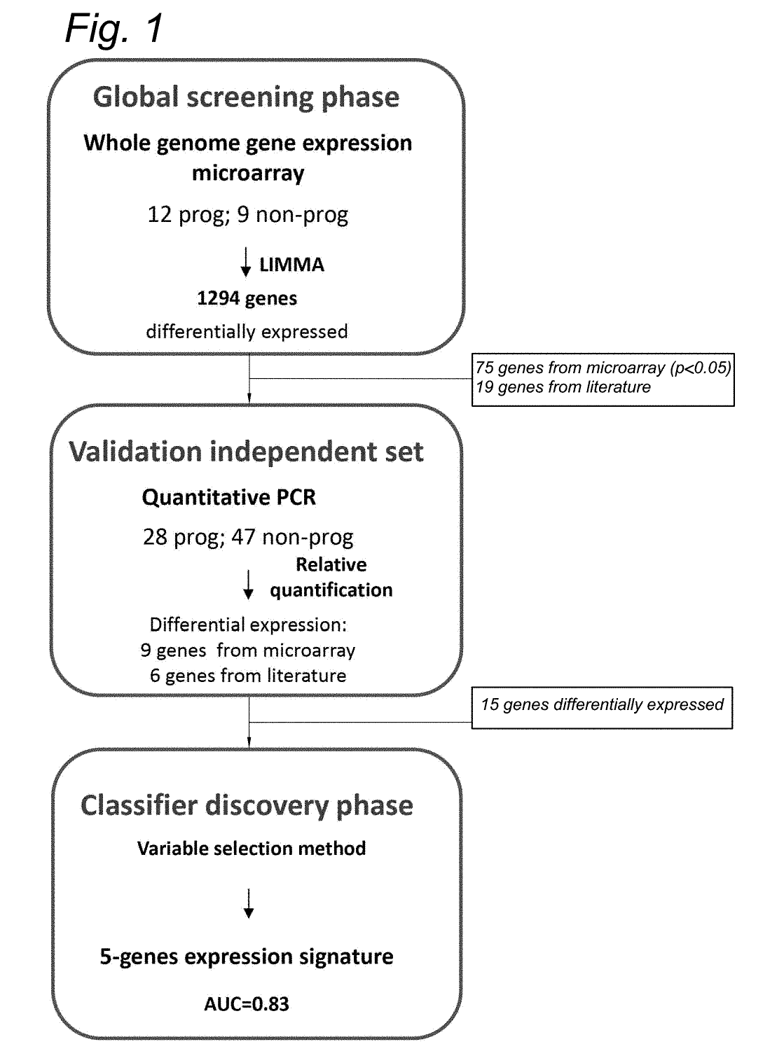

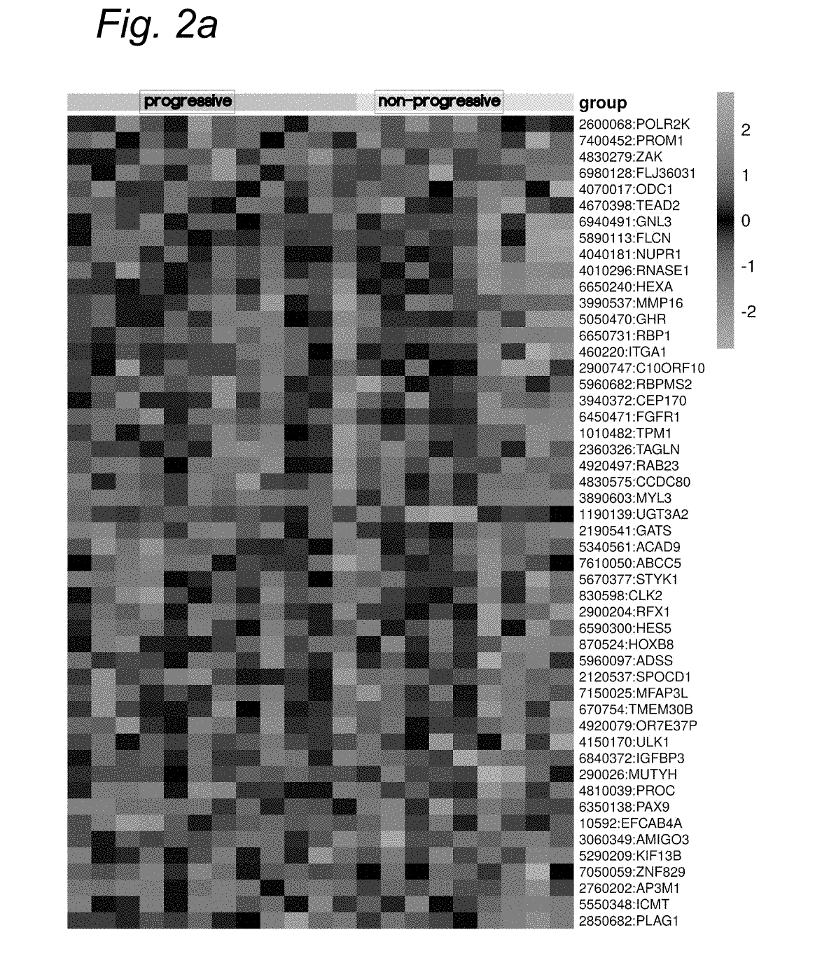

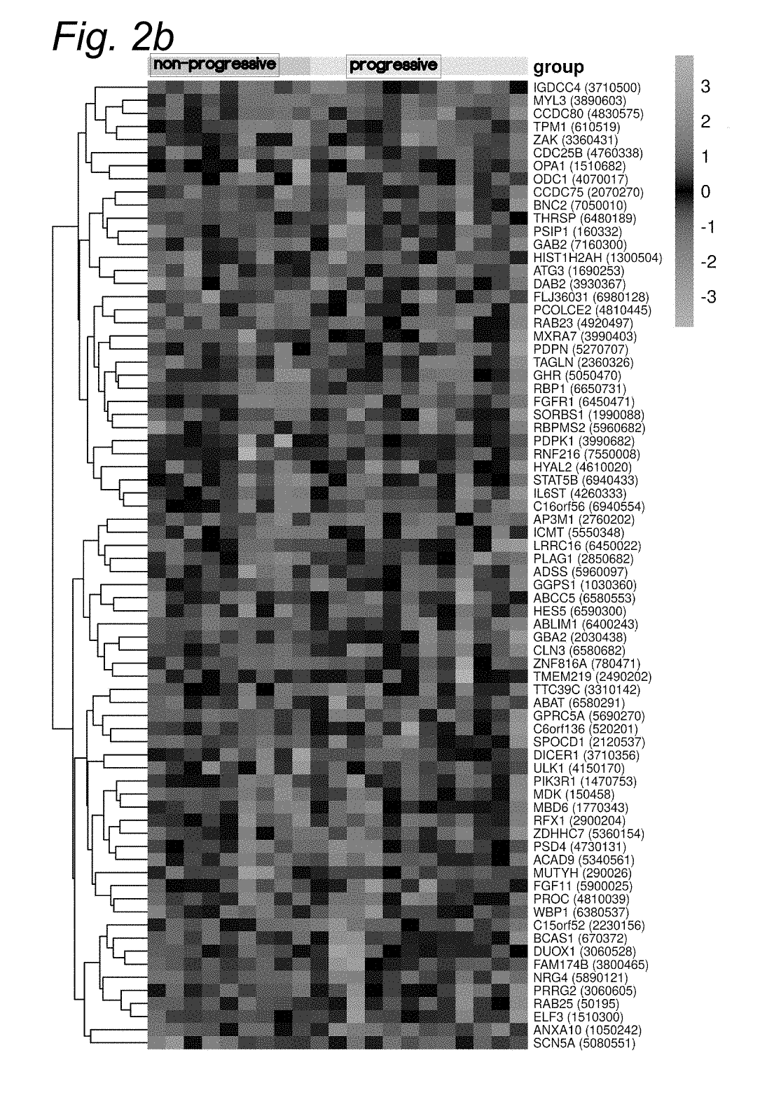

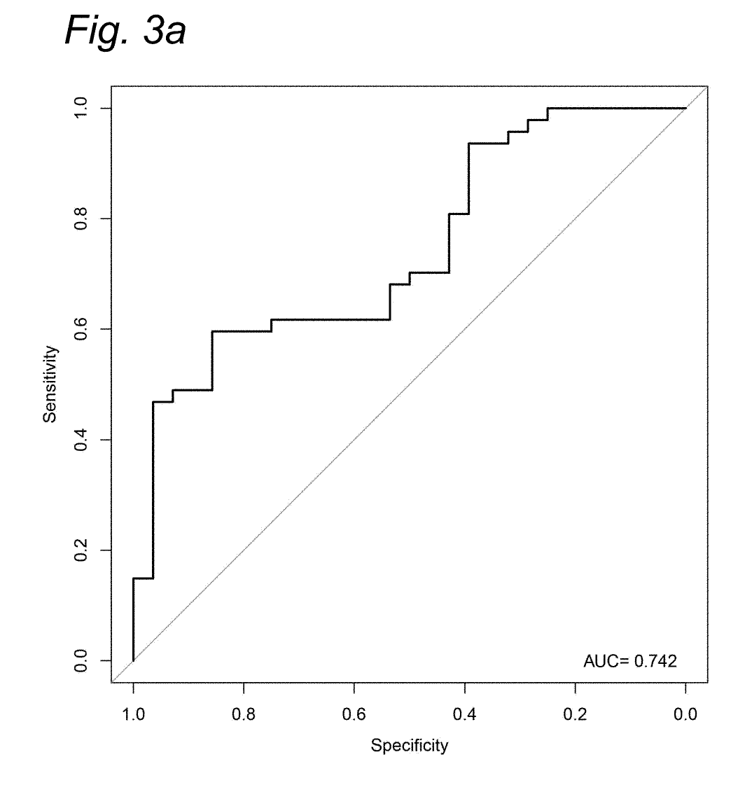

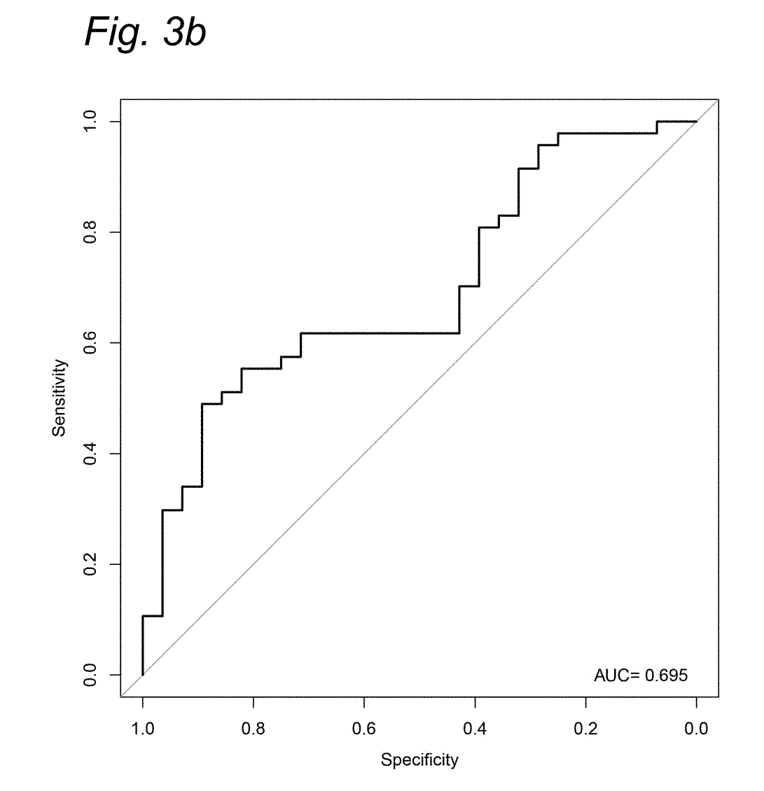

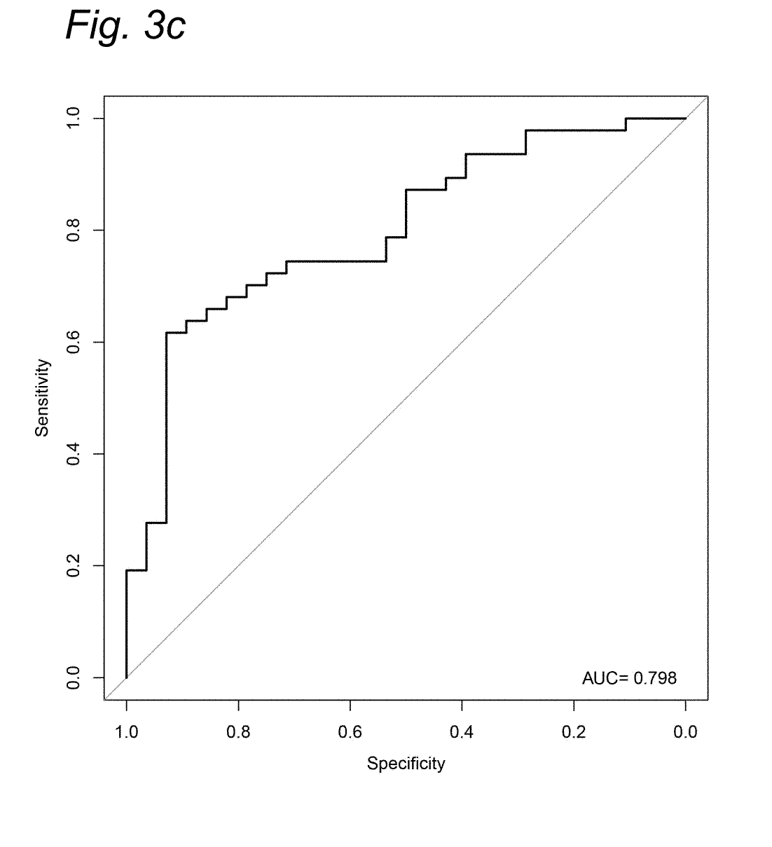

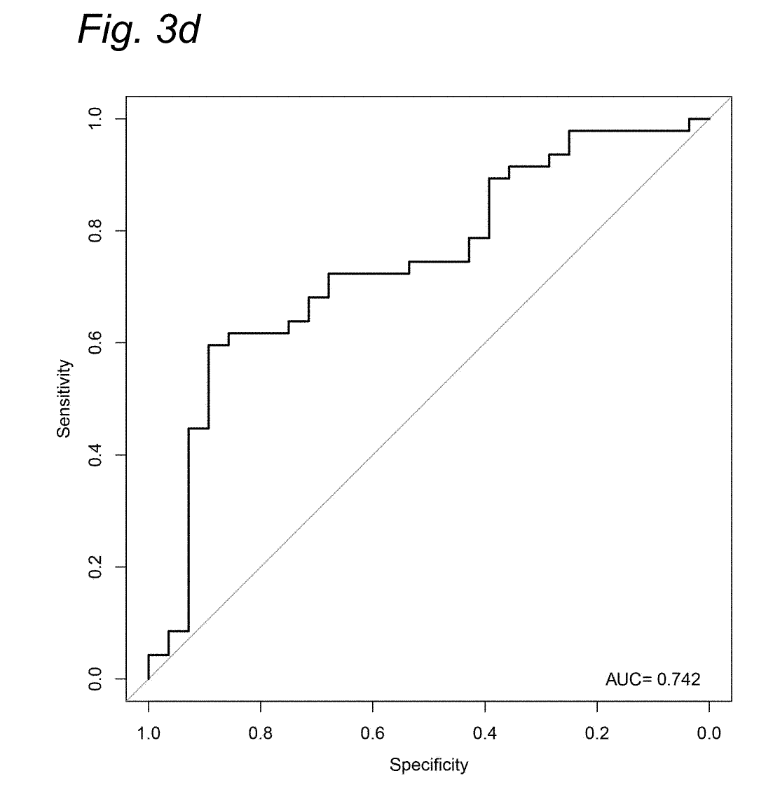

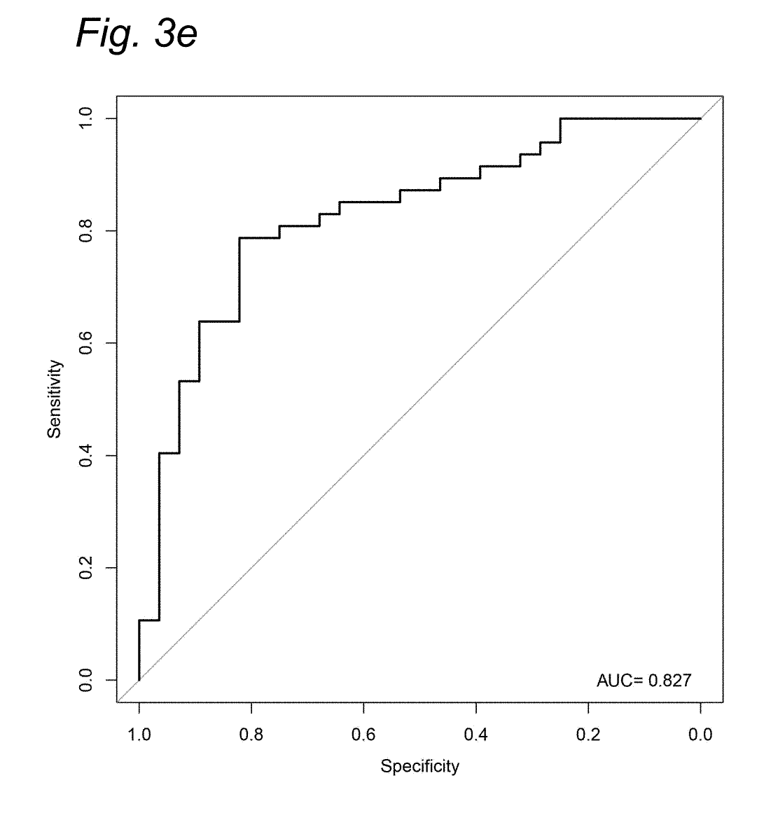

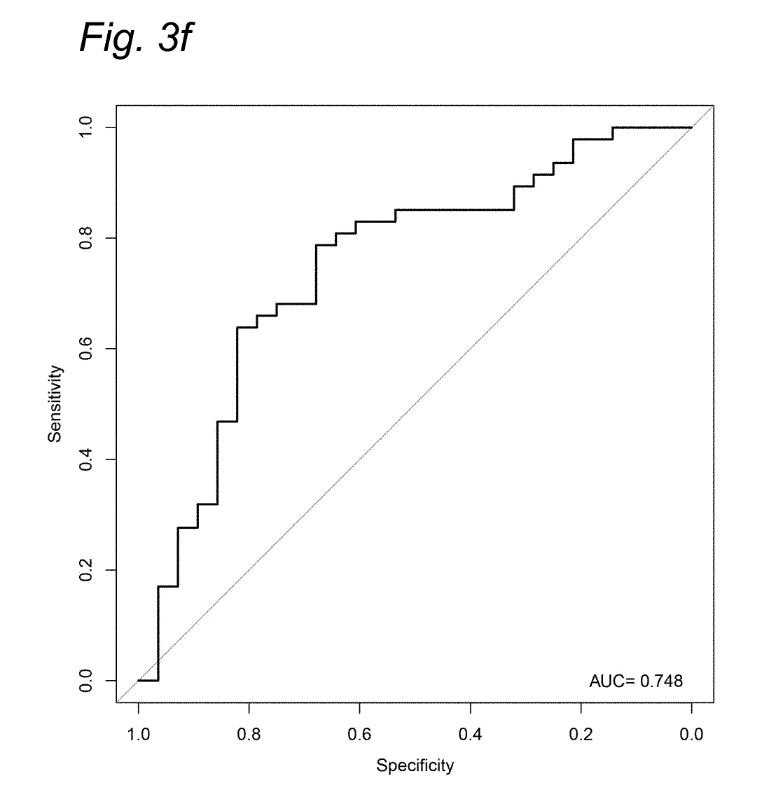

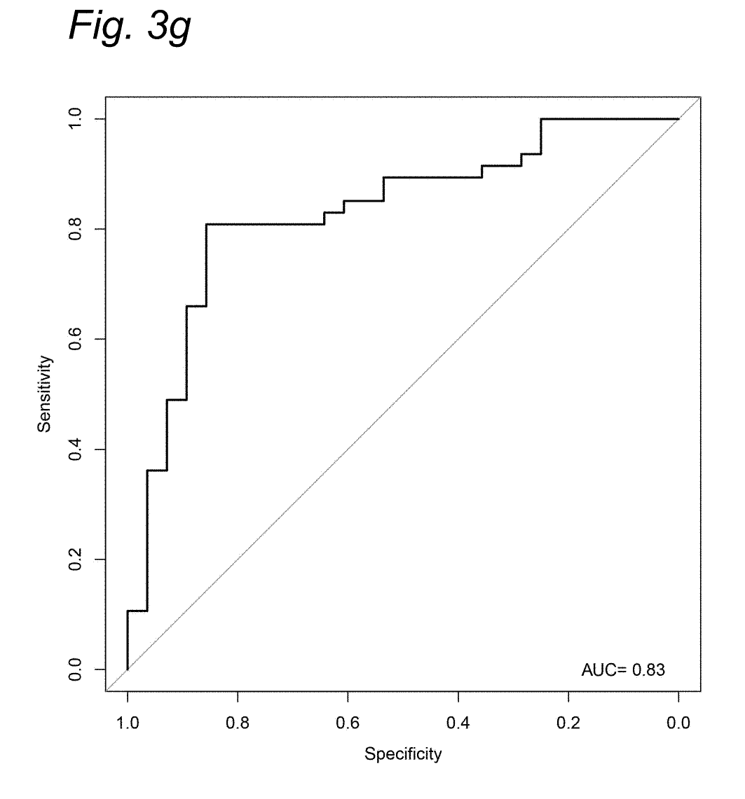

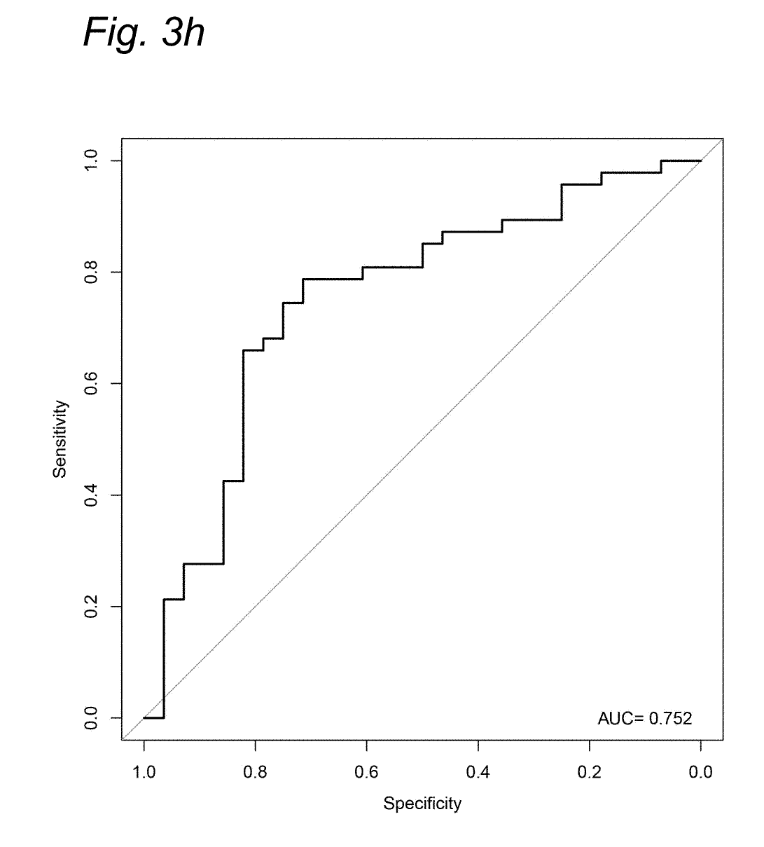

A flowchart of the entire study is shown in FIG. 1. Global mRNA profiling of 21 selected T1G3 BC cases from Hospital Clinic, Barcelona, Spain (12 progressive and 9 non-progressive), was performed using Whole-Genome Gene Expression DASL HT Assay (Illumina, San Diego, Calif., USA), according to manufacturer's instructions [7]. All 21 selected samples had cycle quantification (Cq) values for RPL13A<28, which is considered to be of acceptable RNA quality by the microarray manufacturer's (data not shown).

Data analysis: DASL gene expression data was processed employing quantile normalization using the Lumi bioconductor package. Next, a conservative probe-filtering step excluding those probes with a coefficient of variation of lower than 0.1 was conducted, which resulted in the selection of a total of 22,032 probes (corresponding to 16,653 genes) from the original set of 29,377. For the detection of differentially expressed probes, a linear model was fitted to the data and empirical Bayes moderated statistics were calculated using the Limma package from Bioconductor [8]. Genes representing a change of 1.5-fold or greater and moderated p-value <0.05 were considered as differentially expressed.

### Quantitative PCR Using BioMark 96.96 Dynamic Arrays: Classifier Discovery Phase

Validation of Microarray Expression Data by qPCR in an Additional Set of Samples:

Differential expression of 94 genes was validated in an additional set of 75 tissue samples from two university medical centres (Table 3) using BioMark 96.96 Dynamic Arrays (Fluidigm, South San Francisco, Calif., USA). Seventy-five of 94 genes were selected from microarray expression experiments (p<0.05). Additionally, 19 genes recently described in progressive NMIBC were selected from literature [9-11].

cDNA Synthesis and Pre-Amplification:

cDNA was synthesized from 100 ng of total RNA, using the High Capacity cDNA reverse transcription kit (Applied Biosystems, Foster City, Calif. USA; hereafter referred to as AB) following manufacturer's instructions, except that the final volume of the reaction was 25 μl. Each cDNA sample was used for the multiplex preamplification of the target cDNAs using TaqMan PreAmp Master Mix kit following manufacturer's instructions (AB), except that the final reaction volume of the reaction was 5 μl. A total of 94 target genes and the two endogenous controls (GUSB and PPIA) were used in the pre-amplification reaction.

### Quantitative PCR Using BioMark 96.96 Dynamic Arrays:

cDNA and TaqMan® Gene Expression Assays 20× (AB) were loaded into the Dynamic Array following the manufactures instructions. The real-time quantitative PCR (qPCR) experiments were performed on the BioMark instrument. Primers for cDNA synthesis and PCR were commercially available primers from Life Technologies, see Table 1.

### Quantitative PCR Data Analysis:

The real-time qPCR analysis software was used to obtain cycle quantification (Cq) values. Threshold was manually calculated for each gene. Relative expression levels of target genes within a sample were expressed as ΔCq (ΔCq=Cqendogenous control−Cqtarget gene). The mean Cq value of GUSB and PPIA were used as endogenous control. Genes with Cq values above 35 were considered as lowly expressed, and their ΔCq were imputed to minimum ΔCq value for that gene. Fold change values were generated from the median expression of the genes from the BioMark 96.96 Dynamic Arrays in the groups compared. Differences in gene expression levels between progressive and non-progressive patients were explored using the Student's t test. Significance was defined as p<0.05. R-software was used for all calculations and to construct the heat map.

### Statistical Analysis:

The association of each variable with NMIBC progression status was analyzed by univariate logistic regression. Significance was defined as p values <0.05. Significant transcripts were subjected to a variable selection using elastic net [12] in order to identify the smallest number of transcripts to separate both groups. Since all the samples were used for the model generation, the performance of the model may be over-optimized. To correct this bias, we further performed a leave-one-out cross-validation (LOOCV).

The optimal probability cut-off for the univariate study variables and logistic regression model was computed through a ROC analysis. R-software was used for all calculations. Gene-gene interaction networks for the genes of the model were built by the GeneMANIA Cytoscape 3.0.0 plugin [30]. Physical, co-expression, and pathway gene-gene interactions were evaluated.

### Results

### Global Gene Expression Profile Analysis

The analysis of gene expression profile of 12 progressive and 9 non-progressive T1G3 BC patients by Illumina microarray's resulted in the identification of 1343 transcripts differentially expressed between both groups; 620 down-regulated and 723 up-regulated transcripts in progressive compared with non-progressive cases. Heat map based on the 50 most differentially expressed genes shows a clear distinction between the progressive and the non-progressive groups (FIG. 2).

### Prediction of NMIBC Progression in an Additional Validation Set

To test whether the expression profile of the 94 selected genes (Table 2) can be used to predict progression in T1G3 BC, we additionally analyzed expression of these genes in 28 progressive and 47 non-progressive patients from two hospitals (Table 3) by using BioMark 96.96 Dynamic Arrays.

The relative gene expression values (ΔCq) for the 94 markers in progressive and non-progressive samples were analyzed by univariate analysis to see whether it is possible to separate both groups (Table 2). Fifteen genes were differentially expressed between progressive and non-progressive T1G3 BC samples (Table 4). Univariate analysis shows the performance of each individual biomarker as predictors of NMIBC progression (Table 5).

### Classifier Discovery Phase

To evaluate if a multiplex model could improve performance over single biomarkers, these 15 genes were subjected to a variable selection to identify the minimum combination to accurately predict NMIBC progression. This analysis resulted in a final selection of a five-gene model that contains ANXA10, DAB2, HYAL2, SPOCD1 and MAP4K1. This model is capable of discriminative prediction of progressive from non-progressive T1G3 BC cases with an overall SN of 79% and a SP of 86% (PPV=90%; NPP=71%; ER=19%; AUC=0.83) (FIG. 3). After applying the LOOCV analysis to this five-gene model, SN was 74% for discriminating between both groups of patients with a SP of 79% (PPV=85%; NPP=65%; ER=24%; AUC 0.751) (FIGS. 3 g and h). A gene model comprising SPOCD1 and at least one gene selected from ANXA10, DAB2, HYAL2 and MAP4K1 is, although with somewhat lower SN and SP values also capable to discriminative prediction of progressive from non-progressive T1G3 BC cases (FIGS. 3 a-f).

### ROC; DAB2+SPOCD1: SN of 61.70% and a SP of 60.71% (PPV=72.5%; NPV=48.57%; ER=38.66%; AUC=0.742);

### LOOCV; DAB2+SPOCD1: SN of 61.70% and a SP of 60.72% (PPV=72.5%; NPV=48.57%; ER=38.67%; AUC=0.695); ROC; DAB2+HYAL2+SPOCD1: SN of 72.34% and a SP of 71.42% (PPV=80.95%; NPV=60.61%; ER=28%; AUC=0.798);

### LOOCV; DAB2+HYAL2+SPOCD1: SN of 68.09% and a SP of 67.86% (PPV=78.05%; NPV=55.88%; ER=32%; AUC=0.742); ROC; DAB2+HYAL2+SPOCD1+MAP4K1: SN of 78.72% and a SP of 78.57% (PPV=86.05%; NPV=68.75%; ER=21.33%; AUC=0.827);

### LOOCV; DAB2+HYAL2+SPOCD1+MAP4K1: SN of 68.05% and a SP of 71.43% (PPV=80%; NPV=57.14%; ER=30.67%; AUC=0.748).

The generated network by GeneMANIA shows that there are no direct interactions between the five genes of the model (data not shown).

### DISCUSSION

Prediction of disease outcome for patients diagnosed with high risk NMIBC is an important clinical challenge. Histopathological and clinical tumour characterization is unable to accurately predict whether high risk NMIBC will progress to MIBC. Since treatment regimens change when patients are likely to progress to MIBC, there is a critical need for objective methods to identify those patients. In this study a gene expression signature to distinguish progression in T1G3 BC was identified. For disease progression the expression of a single gene is rarely an indicator since this is regulated by many different mechanisms. Therefore, in the discovery phase an accurate 5-gene signature (ANXA10, DAB2, HYAL2, SPOCD1 and MAP4K1) to predict progression in T1G3 BC was composed. Although LOOCV indicates a certain degree of overfitting, all data obtained after cross validation corroborate the SN and SP for the final model. ANXA10, DAB2, HYAL2 and MAP4K1 had been previously associated with BC, but to the best of our knowledge, this is the first study that relates alterations in expression of SPOCD1 (SpenParalog and OrthologsC-terminal domain-containing 1) with BC. It should be noted that no direct interactions between the five genes were found (data not shown).

SPOCD1 encodes a protein that belongs to the TFIIS family of transcription factors and the 8-fold down-regulation that was observed may lead to a different transcription profile.

ANXA10 (Annexin A10) is a calcium and phospholipid-binding protein that participates in cell differentiation and tissue growth. ANXA10 was 3-fold down-regulated in progressive T1G3 BC patients. This is in concordance with previous data in which ANXA10 down-regulation correlated with increased invasion, proliferation and metastasis in colorectal, gastric and lung cancer cell lines [13-15]. Dyrskjot et al. studied the ANXA10 gene expression in BC. In 150 patients a gene expression signature with among others ANXA10 was able to predict the presence of CIS, and progression to MIBC [3]. Subsequently, Munksgaard et al. focused on low ANXA10 mRNA expression and showed that this was associated with the presence of concomitant CIS and a shorter progression free survival. Furthermore, they found in two BC cell lines that ANXA10 down-regulation induced an increase of proliferation and migration [16].

DAB2, disabled 2, mitogen-responsive phosphoprotein, plays a pivotal role in cellular homeostasis. It modulates protein trafficking, cytoskeleton organization, cell adhesion and migration, and cell signaling of various receptor tyrosine kinases. Results about DAB2 expression levels in cancer appear to be conflicting as up-regulation as well as down-regulation of DAB2 has been reported. A systematic review showed that DAB2 was down-regulated in 70 to 90% of human cancers, including breast cancer and nasopharyngeal carcinomas [17], whereas other studies showed a weak to moderate positive DAB2 immunostaining in lung and esophageal cancer [18,19]. For BC moderate expression of DAB2 was observed in 60% of the transitional cell lines used [20], but Karam et al showed that DAB2 expression decreased in comparison to normal bladder and that decreased DAB2 expression was more prominent in advanced (Ta, Tis and T1 versus T2-4) BC [21].

In our study DAB2 was 3-fold up-regulated in progressive NMIBC patients compared to non-progressive NMIBC patients. Clearly, our patient population differs as we specifically studied pT1G3 BC tumours and excluded patients with concomitant CIS, which may explain this seeming inconsistency.

HYAL2, Hyaluronidase 2, degrades hyaluronan into smaller fragments endowed with specific biological activities such as inflammation and angiogenesis. The exact role in tumour promotion or suppression is not clear yet, but HYAL2 plays a role in tumour cell adhesion and migration. Previously, elevated hyaluronidase levels were described in urine of bladder and prostate cancer patients and therefore it was considered a biomarker for these cancers [21,23]. In progressive T1G3 BC patients HYAL2 was 3-fold up-regulated. This is in concordance with recently published data on colorectal cancer, in which HYAL2 was overexpressed, especially in advanced stages [24]. Furthermore, of HYAL2 overexpression correlated with deeper invasiveness of breast cancer cell lines [25].

MAP4K1 is a mitogen-activated protein 4 kinase 1 also known as HPK1 (Hematopoietic Progenitor Kinase 1), which regulates cell cycle, cell adhesion, migration and apoptosis. MAP4K1 is activated by a range of environmental stimuli, including genotoxic stress, growth factors, inflammatory cytokines and antigen receptor triggering. MAP4K1 was 1.5-fold up-regulated in our cohort of progressive patients. Wang and co-workers showed in BC cell lines (T24 and 5637) also an up-regulated expression of MAP4K1 [26]. MAP4K1 can be activated after being phosphorylated by tyrosine kinase and in turn may activate the Raf→MEK1→MAPK/ERK pathway to modulate the cell cycle, cell adhesion, migration and apoptosis [27]. Although the expression of MAP4K1 is up-regulated in BC, the process of its phosphorylation may be inhibited, thus blocking the MAPK/ERK pathway [26].

In a comprehensive, integrated study of 131 MIBC four expression subtypes were distinguished [28]. Type 1 MIBC was enriched with papillary features supporting the idea that these MIBC correspond to the papillary NMIBC that have shown progression. Whether our gene signature of progressive T1G3 BC is comparable to type 1 MIBC deserves further study.

Given the genomic heterogeneity of BC, one limitation of this study is its sample size. Furthermore, our analysis may have underestimated the genetic variation present within individual tumours as we sampled only one segment of the tumour. In addition, although studies in two independent cohorts of patients showed comparable results, a final validation in an independent larger series is needed for clinical implementation. Another limitation is the retrospective non-randomized character of this study. However, prospective recruitment of patients is very difficult because the percentage of progressive T1G3 BC is low and the follow up needs to be relatively long to observe progression; in our patient population the longest time interval to progress to MIBC was 81 months.

An important benefit of this study is the fact that the methodology used is widely available and thus this 5-gene model should be easily implemented in clinical practice. Quantitative PCR is reasonably simple to perform and is inexpensive. Identification of new genes associated with a high probability of tumour progression in combination with a robust, easy-to-use and reliable algorithm may contribute to tailor treatment and surveillance strategies in these patients.

### CONCLUSIONS

This study describes a 5-gene expression signature that is capable to identify high risk NMIBC patients that progress to MIBC. The utilization of this prognostic technique next to the available clinical and histopathological data will enable a more personalized therapy. Patients with a high risk of progression can be treated more radically. By means of logistic regression we have demonstrated that two or more of these five genes not only complement each other but show synergy. Thus, evidence is provided of an independent added value.

### Tables

TABLE 2
Univariate analysis of predictors for tumour progression in T1G3
BC patients.

| GENE | | | | |
|---|---|---|---|---|
| YMBOL | OR | 95% | CI | p |
| ABAT | 0.91 | 1.35 | 0.62 | 0.6417 |
| ABCC5 | 1.29 | 1.95 | 0.86 | 0.2211 |
| ABLIM1 | 1.59 | 2.56 | 0.99 | 0.0553 |
| ACAD9 | 1.01 | 1.57 | 0.65 | 0.9563 |
| ADSS | 1.33 | 2.06 | 0.85 | 0.2084 |
| ANXA10 | 1.61 | 2.22 | 1.17 | 0.0037* |
| AP3M1 | 1.40 | 2.19 | 0.89 | 0.1442 |
| ATG3 | 0.99 | 2.05 | 0.48 | 0.9834 |
| BCAS1 | 1.41 | 2.04 | 0.97 | 0.0750 |
| BIRC4^{§} | 1.15 | 1.94 | 0.68 | 0.5928 |
| BIRC5^{§} | 1.11 | 1.87 | 0.66 | 0.6946 |
| BIRC6^{§} | 1.21 | 1.98 | 0.74 | 0.4367 |
| BNC2 | 0.53 | 0.88 | 0.32 | 0.0134* |
| BNIP3L^{§} | 1.54 | 2.28 | 1.04 | 0.0330* |
| C15orf52 | 1.16 | 1.92 | 0.70 | 0.5752 |
| C16orf56 | 0.84 | 1.25 | 0.56 | 0.3855 |
| C16orf74^{§} | 1.70 | 2.74 | 1.05 | 0.0294* |
| C6orf136 | 1.17 | 1.72 | 0.79 | 0.4408 |
| CCDC75 | 1.09 | 1.96 | 0.61 | 0.7772 |
| CCDC80 | 0.78 | 1.06 | 0.58 | 0.1096 |
| CDC20^{§} | 0.77 | 1.39 | 0.43 | 0.3844 |
| CDC25B | 0.61 | 1.01 | 0.37 | 0.0552 |
| CLN3 | 1.02 | 1.99 | 0.52 | 0.9558 |
| CSRP2^{§} | 0.91 | 1.17 | 0.71 | 0.4668 |
| DAB2 | 0.40 | 0.74 | 0.22 | 0.0031* |
| DICER1 | 1.37 | 2.29 | 0.82 | 0.2316 |
| DUOX1 | 1.13 | 2.01 | 0.63 | 0.6913 |
| ELF3 | 1.27 | 1.73 | 0.93 | 0.1289 |
| ERGIC2 | 0.86 | 1.24 | 0.59 | 0.4069 |
| FAM174B | 1.36 | 2.27 | 0.81 | 0.2447 |
| FGF11 | 1.17 | 1.80 | 0.76 | 0.4745 |
| FGFR1 | 0.60 | 0.93 | 0.39 | 0.0231* |
| FGFR3^{§} | 1.43 | 2.26 | 0.91 | 0.1189 |
| FLJ36031 | 0.55 | 0.89 | 0.33 | 0.0163* |
| GAB2 | 0.67 | 1.05 | 0.43 | 0.0794 |
| GBA2 | 1.53 | 2.64 | 0.89 | 0.1268 |
| GGPS1 | 1.16 | 1.80 | 0.75 | 0.4963 |
| GHR | 0.70 | 1.07 | 0.46 | 0.1018 |
| GPRC5A | 1.31 | 2.03 | 0.84 | 0.2363 |
| GRB7^{§} | 1.27 | 1.93 | 0.83 | 0.2660 |
| HES5 | 0.64 | 0.96 | 0.43 | 0.0329* |
| HIST1H2AH | 0.46 | 0.94 | 0.23 | 0.0333* |
| HIST2H4B^{§} | 0.65 | 0.99 | 0.43 | 0.0425* |
| HYAL2 | 0.40 | 0.78 | 0.20 | 0.0070* |
| ICMT | 0.69 | 1.15 | 0.41 | 0.1538 |
| IGDCC4 | 0.71 | 1.06 | 0.47 | 0.0912 |
| IGF2^{§} | 0.75 | 1.26 | 0.45 | 0.2809 |
| IL6ST | 0.77 | 1.21 | 0.50 | 0.2589 |
| LRRC16 | 1.23 | 2.04 | 0.74 | 0.4352 |
| MAP4K1^{§} | 0.44 | 0.75 | 0.25 | 0.0030* |
| MBD6 | 0.93 | 1.50 | 0.57 | 0.7616 |
| MCM7^{§} | 0.96 | 1.86 | 0.50 | 0.9020 |
| MDK | 0.69 | 1.19 | 0.40 | 0.1786 |
| MUTYH | 0.60 | 0.96 | 0.37 | 0.0347* |
| MXRA7 | 0.68 | 1.19 | 0.39 | 0.1808 |
| MYL3 | 0.71 | 1.43 | 0.35 | 0.3385 |
| NCKAP1^{§} | 1.07 | 1.97 | 0.58 | 0.8385 |
| NRG4 | 1.57 | 2.67 | 0.92 | 0.0953 |
| ODC1 | 0.84 | 1.51 | 0.47 | 0.5638 |
| OPA1 | 1.04 | 1.72 | 0.63 | 0.8639 |
| PCOLCE2 | 0.80 | 1.08 | 0.60 | 0.1492 |
| PDPK1 | 0.56 | 1.07 | 0.30 | 0.0799 |
| PDPN | 1.06 | 1.54 | 0.73 | 0.7554 |
| PFKFB4^{§} | 1.66 | 2.67 | 1.03 | 0.0386* |
| PIK3R1 | 1.02 | 1.69 | 0.61 | 0.9488 |
| PLAG1 | 1.29 | 2.53 | 0.65 | 0.4644 |
| PPARD^{§} | 1.70 | 2.78 | 1.04 | 0.0332* |
| PROC | 0.86 | 1.55 | 0.48 | 0.6201 |
| PRRG2 | 1.29 | 2.45 | 0.68 | 0.4274 |
| PSD4 | 1.12 | 1.82 | 0.69 | 0.6376 |
| PSIP1 | 1.26 | 1.95 | 0.81 | 0.2991 |
| RAB23 | 0.69 | 1.13 | 0.42 | 0.1426 |
| RAB25 | 1.54 | 2.38 | 0.99 | 0.0543 |
| RBP1 | 0.62 | 1.00 | 0.39 | 0.0520 |
| RBPMS2 | 0.66 | 1.05 | 0.41 | 0.0794 |
| RFX1 | 1.71 | 3.03 | 0.97 | 0.0637 |
| RNF216 | 1.22 | 1.80 | 0.82 | 0.3281 |
| SCN5A | 1.16 | 1.70 | 0.79 | 0.4522 |
| SERPINB5^{§} | 1.65 | 2.40 | 1.14 | 0.0083* |
| SORBS1 | 0.98 | 1.38 | 0.69 | 0.8988 |
| SPOCD1 | 1.85 | 2.81 | 1.21 | 0.0043* |
| STAT5B | 0.56 | 1.03 | 0.31 | 0.0615 |
| TAGLN | 0.79 | 1.07 | 0.58 | 0.1257 |
| THRSP | 1.31 | 2.08 | 0.82 | 0.2605 |
| TMEM219 | 1.26 | 1.83 | 0.86 | 0.2344 |
| TPM1 | 1.39 | 2.07 | 0.94 | 0.1002 |
| TRAF4^{§} | 1.35 | 2.19 | 0.83 | 0.2245 |
| TTC39C | 1.15 | 2.06 | 0.64 | 0.6420 |
| ULK1 | 1.50 | 2.39 | 0.95 | 0.0851 |
| WBP1 | 0.80 | 1.00 | 0.64 | 0.0482* |
| WHSC1^{§} | 1.36 | 2.43 | 0.77 | 0.2924 |
| ZAK | 0.94 | 1.55 | 0.57 | 0.8173 |
| ZDHHC7 | 2.85 | 7.19 | 1.13 | 0.0261* |
| ZNF816A | 1.18 | 1.89 | 0.73 | 0.4952 |

*p <0.05
^{§}Gene selected from literature

TABLE 3
Demographic and clinical characteristics of enrolled patients

| | | Discovery | Discovery |
|---|---|---|---|
| | Screening phase | phase | phase |
| | Hospital Clinic | Hospital Clinic | Radboud UMC |
| | Barcelona | Barcelona | Nijmegen |
| Progressive patients | | | |
| Male | 11 | 8 | 12 |
| Female | 1 | 4 | 4 |
| Mean age | 74 (54-86) | 74 (57-90) | 66 (41-83) |
| Subtotals | 12 | 12 | 16 |
| Non-progressive | | | |
| patients | | | |
| Male | 8 | 18 | 15 |
| Female | 1 | 8 | 6 |
| Mean age | 67 (40-83) | 72 (47-85) | 61(39-77) |
| Subtotals | 9 | 26 | 21 |
| Totals | 21 | 38 | 37 |

TABLE 4
Comparison of microarray and qPCR results for the 15 genes found
differentially expressed in qPCR

| GENE | Microarrays | | qPCR | |
|---|---|---|---|---|
| SYMBOL | FOLD CHANGE | p | FOLD CHANGE | p |
| ANXA10 | −3.09 | 0.041* | −3.42 | 0.002* |
| BNC2 | 2.38 | 0.004* | 2.37 | 0.006* |
| BNIP3L^{§} | −1.27 | 0.600 | −1.79 | 0.037* |
| C16orf74^{§} | 1.07 | 0.868 | −1.63 | 0.028* |
| DAB2 | 2.85 | 0.003* | 1.79 | 0.001* |
| FGFR1 | 12.07 | 0.001* | 1.62 | 0.019* |
| FLJ36031 | 2.16 | 0.002* | 1.70 | 0.012* |
| HIST1H2AH | 2.78 | 0.006* | 1.32 | 0.035* |
| HYAL2 | 2.65 | 0.003* | 1.80 | 0.006* |
| MAP4K1^{§} | 1.44 | 0.164 | 2.05 | 0.003* |
| PFKFB4^{§} | 1.39 | 0.337 | −1.66 | 0.026* |
| PPARD^{§} | −1.07 | 0.784 | −1.62 | 0.026* |
| SERPINB5^{§} | −2.18 | 0.084 | −2.28 | 0.007* |
| SPOCD1 | −8.00 | 0.000* | −2.25 | 0.003* |
| ZDHHC7 | −2.40 | 0.004* | −1.27 | 0.017* |

*p <0.05;
^{§}Gene selected from literature
Fold change values were generated from the mean expression of the mRNAs in the groups compared.

TABLE 3
(5). Univariate analysis of the individual biomarkers
as predictors of NMIBC progression

| GENE | | | | speci- | sensi- | | |
|---|---|---|---|---|---|---|---|
| SYMBOL | OR | p-value | AUC | ficity | tivity | NPV | PPV |
| ANXA10 | 1.61 | 0.004 | 0.76 | 0.72 | 0.77 | 0.64 | 0.83 |
| BNC2 | 0.53 | 0.013 | 0.73 | 0.75 | 0.61 | 0.56 | 0.79 |
| BNIP3L | 1.54 | 0.033 | 0.66 | 0.75 | 0.55 | 0.50 | 0.79 |
| C16orf74 | 1.70 | 0.029 | 0.70 | 0.79 | 0.62 | 0.56 | 0.82 |
| DAB2 | 0.40 | 0.003 | 0.74 | 0.71 | 0.74 | 0.63 | 0.81 |
| FGFR1 | 0.60 | 0.023 | 0.64 | 0.65 | 0.53 | 0.44 | 0.74 |
| FLJ36031 | 0.55 | 0.016 | 0.69 | 0.68 | 0.67 | 0.58 | 0.76 |
| HIST1H2AH | 0.46 | 0.033 | 0.66 | 0.67 | 0.65 | 0.53 | 0.77 |
| HYAL2 | 0.40 | 0.007 | 0.75 | 0.77 | 0.72 | 0.61 | 0.85 |
| MAP4K1 | 0.44 | 0.003 | 0.75 | 0.78 | 0.61 | 0.55 | 0.82 |
| PFKFB4 | 1.66 | 0.039 | 0.64 | 0.59 | 0.68 | 0.53 | 0.73 |
| PPARD | 1.70 | 0.033 | 0.64 | 0.57 | 0.70 | 0.53 | 0.73 |
| SERPINB5 | 1.65 | 0.008 | 0.71 | 0.78 | 0.66 | 0.57 | 0.84 |
| SPOCD1 | 1.85 | 0.004 | 0.73 | 0.63 | 0.72 | 0.57 | 0.77 |
| ZDHHC7 | 2.85 | 0.026 | 0.64 | 0.71 | 0.57 | 0.50 | 0.77 |

### REFERENCE LIST

1. Sylvester R J, van der Meij den AP, Oosterlinck W, et al. Predicting recurrence and progression in individual patients with stage Ta T1 bladder cancer using EORTC risk tables: a combined analysis of 2596 patients from seven EORTC trials. Eur Urol 2006; 49:466-5.
2. Modlich O, Prisack H B, Pitschke G, et al. Identifying superficial, muscle-invasive, and metastasizing transitional cell carcinoma of the bladder: use of cDNA array analysis of gene expression profiles. Clin Cancer Res 2004; 10:3410-21.
3. Dyrskjot L, Zieger K, Real F X, et al. Gene expression signatures predict outcome in non-muscle-invasive bladder carcinoma: a multicenter validation study. Clin Cancer Res 2007; 13:3545-51.
4. Mengual L, Burset M, Ars E, et al. DNA microarray expression profiling of bladder cancer allows identification of noninvasive diagnostic markers. J Urol 2009; 182:741-8.
5. Lopez-Beltran, A., Sauter, G., Gasser, T. et al.: Tumours of the Urinary System. In: Pathology and Genetics of Tumours of the Urinary System and Male Genital Organs. World Health Organization Classification of Tumours. Lyon: IARC Press; 2004. p. 89.
6. Sobin, L. H., Gospodariwicz, M., and Wittekind, C. H.: TNM Classification of Malignant Tumours. UICC International Union Against Cancer. New York: Wiley-Blackwell; 2009. p. 243-248.
7. Fan J B, Gunderson K L, Bibikova M, et al. Illumina universal bead arrays. Methods Enzymol 2006; 410:57-73.
8. Smyth G K. Limma: linear models for microarray data. In: Bioinformatics and Computational Biology Solutions using R and Bioconductor. Springer; 2005. p. 397-420.
9. Dyrskjot L, Zieger K, Kruhøffer M, et al. A molecular signature in superficial bladder carcinoma predicts clinical outcome. Clin Cancer Res 2005; 11:4029-36.
10. Yun S J, Jo S W, Ha Y S, et al. PFKFB4 as a prognostic marker in non-muscle-invasive bladder cancer. Urol Oncol 2012; 30:893-9.
11. Kim W T, Yun S J, Park C, et al. Identification of C16orf74 as a marker of progression in primary non-muscle invasive bladder cancer. PLoS One 2010; 5:e15260.
12. Zou, H. and Hastie, T.: Regularization and variable selection via the elastic net. Journal of the Royal Statistical Society: Series B (Statistical Methodology) 2005; 67:199.
13. Patsos G, Germann A, Gebert J, Dihlmann S. Restoration of absent in melanoma 2(AIM2) induces G2/M cell cycle arrest and promotes invasion of colorectal cancer cells. Int J Cancer 2010; 126:1838-49.
14. Kim J, Kim M A, Jee C D, Jung E J, Kim W H. Reduced expression and homozygous deletion of annexin A10 in gastric carcinoma. Int J Cancer 2009; 125:1842-50.
15. Matsubara D, Niki T, Ishikawa S, et al. Differential expression of S100A2 and S100A4 in lung adenocarcinomas: clinicopathological significance, relationship to p53 and identification of their target genes. Cancer Sci 2005; 96:844-57.
16. Munksgaard P P, Mansilla F, Brems Eskildsen A S, et al. Low ANXA10 expression is associated with disease aggressiveness in bladder cancer. Br J Cancer 2011; 105:1379-87.
17. Zhang Z, Chen Y, Tang J, Xie X. Frequent loss expression of dab2 and promotor hypermethylation in human cancers: a meta-analysis and systematic review. Pak J Med Sci 2014; 30:432-7.
18. Xu H T, Yang L H, Li Q C, et al. Disabled-2 and Axin are concurrently colocalized and underexpressed in lung cancers. Hum Pathol 2011; 42:1491-8.
19. Anupam K, Tusharkant C, Gupta S D, Ranju R. Loss of disabled-2 expression is an early event in esophageal squamous tumorigenesis. World J Gastroenterol 2006; 12:6041-5.
20. Zhou J, Hernandez G, Tu S W, et al. Synergistic induction of DOC-2/DAB2 gene expression in transitional cell carcinoma in the presence of GATA6 and histone deacetylase inhibitor. Cancer Res 2005; 65:6089-96.
21. Karam J A, Shariat S F, Huang H Y, et al. Decreased DOC-2/DAB2 expression in urothelial carcinoma of the bladder. Clin Cancer Res 2007; 13:4400-6.
22. Lokeshwar V B, Lokeshwar B L, Pham H T, Block N L. Association of elevated levels of hyaluronidase, a matrix-degrading enzyme, with prostate cancer progression. Cancer Res 1996; 56:651-7.
23. Konety B R, Getzenberg R H. Urine based markers of urological malignancy. J Urol 2001; 165:600-11.
24. Bouga H, Tsouros I, Bounias D, et al. Involvement of hyaluronidases in colorectal cancer. BMC Cancer 2010; 10:499.
25. Udabage L, Brownlee G R, Nilsson S K, Brown T J. The over-expression of HAS2, Hyal-2 and CD44 is implicated in the invasiveness of breast cancer. Exp Cell Res 2005; 310:205-17.
26. Wang Y, Luo H, Li Y, Chen T, Wu S, Yang L. hsa-miR-96 up-regulates MAP4K1 and IRS1 and may function as a promising diagnostic marker in human bladder urothelial carcinomas. Mol Med Rep 2012; 5:260-5.
27. Oehrl W, Kardinal C, Ruf S, et al. The germinal center kinase (GCK)-related protein kinases HPK1 and KHS are candidates for highly selective signal transducers of Crk family adapter proteins. Oncogene 1998; 17:1893-901.
28. Cancer Genome Atlas Research Network. Comprehensive molecular characterization of urothelial bladder carcinoma. Nature 2014; 507:315-22.
29. M. Babjuk (Chair), A. Bohle, M. Burger, E. Compérat, E. Kaasinen, J. Palou, M. Rouprêt, B. W. G. van Rhijn, S. Shariat, R. Sylvester, and R. Zigeuner. Guidelines on Non-muscle-invasive Bladder Cancer (TA, T1 and CIS), Bajuk et al, European Association of Urology 2015.
30. Montojo J, Zuberi K, Rodriguez H, Bader G D, and Morris Q. GeneMANIA: fast gene network construction and function prediction for cytoscape. F1000Res 2014; 3:153.

1.-15. (canceled)

16. A method for the prediction of progression of bladder cancer in a subject comprising determination of the expression level of at least one, two, three, four or five bladder cancer marker genes selected from the group consisting of ANXA10, BNC2, BNIP3L, C16orf74, DAB2, FGFR1, FLJ36031, HIST1H2AH, HYAL2, MAP4K1, PFKFB4, PPARD, SERPINB5, SPOCD1 and ZDHHC7, in a sample from a subject, wherein differential expression of at least one, two, three, four or five marker genes is predictive for progression of the bladder cancer.

17. A method according to claim 16, wherein differential expression is determined:
by comparing the expression level of the bladder cancer marker gene in a sample from a subject suffering from bladder cancer to the expression level of the marker gene in a corresponding sample from a subject not suffering from bladder cancer, or,
by comparing the expression level of the bladder cancer marker gene in a sample from a subject suffering from bladder cancer to an expression level reference value of the bladder cancer marker gene that is indicative for progression of the bladder cancer, or,
by comparing the expression level of the bladder cancer marker gene in a sample from a subject suffering from bladder cancer to an expression level reference value of the bladder cancer marker gene that is indicative for non-progression of the bladder cancer, or,
by comparing the expression level of the bladder cancer marker gene in a sample from a subject suffering from bladder cancer to the expression level of the bladder cancer marker gene in a corresponding sample from a subject suffering from bladder cancer that later in time demonstrated progression of the bladder cancer (i.e. a progressive subject), or
by comparing the expression level of the bladder cancer marker gene in a sample form a subject suffering from bladder cancer to the expression level of the bladder cancer marker gene in a corresponding sample from a subject suffering from bladder cancer that later in time did not demonstrate progression of the bladder cancer (i.e. a non-progressive subject).

18. A method according to claim 16, further comprising determination of the expression level of an endogenous control gene.

19. A method according to claim 16, wherein the endogenous control gene is selected from the group consisting of GUSB and PPIA.

20. A method according to claim 16, wherein differential expression of a bladder cancer marker gene is defined as at least 10%, 20%, 30%, 50%, 60%, 70%, 80%, 90%, 100%, 150%, 200%, 300%, 400%, 500%, 600%, 700% or at least 800% up-regulation or down-regulation, compared to the expression level of the corresponding bladder cancer marker gene in a corresponding sample from a non-progressive subject, the expression level of the corresponding bladder cancer marker gene in a corresponding sample from a subject not suffering from bladder cancer, the expression level reference value of the bladder cancer marker gene that is indicative for non-progression of the bladder cancer, or the expression level reference value of the bladder cancer marker gene that is indicative progression of the bladder cancer.

21. A method according to claim 16, wherein the progression of bladder cancer is the progression from non-muscle-invasive bladder cancer to muscle-invasive bladder cancer.

22. A method according to claim 16, wherein the expression level of at least two bladder cancer marker genes selected from the group consisting of ANXA10, BNC2, BNIP3L, C16orf74, DAB2, FGFR1, F1136031, HIST1H2AH, HYAL2, MAP4K1, PFKFB4, PPARD, SERPINB5, SPOCD1 and ZDHHC7 is determined wherein one of the at least two bladder cancer marker genes is SPOCD1.

23. A method according to claim 22, wherein the expression level of at least ANXA10, DAB2, HYAL2, MAP4K1 and SPOCD1 is determined.

24. A method according to claim 16, wherein the sample is a tumor sample.

25. A method according to claim 16, wherein the determination of the expression level is performed by a nucleic acid amplification method.

26. A method according to claim 25, wherein the nucleic acid amplification method is multiplex PCR.

27. A method according to claim 16, wherein the method is an in vitro or ex vivo method.

28. A method according to claim 16, wherein the sample is a bladder fluid sample.

29. A method according to claim 28, wherein the bladder fluid sample is urine.

30. A method of treatment comprising:
establishing a prediction of progression of bladder cancer in a subject using a method according to claim 16, and,
treating a subject wherein the bladder cancer is predicted to progress for progressive bladder cancer, or,
treating a subject wherein the bladder cancer is predicted not to progress for non-progressive bladder cancer.

31. A method of treatment according to claim 30, wherein treatment of a subject wherein the bladder cancer is predicted to progress, comprises cystectomy, or wherein treatment of a subject wherein the bladder cancer is predicted not to progress comprises intravesical therapy such as immune therapy using BCG, interferon, chemotherapy or hypothermia.

32. A method of treatment according to claim 31, wherein the chemotherapy uses mitomycin, thiotepa, valrubicin, doxorubicin or gemcitabine.

33. A kit for performing the method according to claim 16, comprising at least one, two, three, four or five sets of nucleic acid molecules suitable for the determination of the expression level of a bladder cancer marker gene as defined in claim 16.

34. A kit according to claim 33, wherein the at least one, two, three, four or five sets of nucleic acid molecules suitable for the determination of the expression level of a bladder cancer marker gene as defined in of claim 16 are suitable for a nucleic acid amplification technique.

35. A kit according to claim 33, comprising a composition comprising at least one, two, three, four or five sets of nucleic acid molecules suitable for the determination of the expression level of a bladder cancer marker gene as defined in claim 16 by a nucleic acid amplification technique.
